(19) United States
(12) Patent Application Publication
(10) Pub. No.: US 2013/0230885 A1
(43) Pub. Date: Sep. 5, 2013

(54) LOV-DOMAIN PROTEIN FOR PHOTOSENSITIVE DEFUNCTIONALIZATION

(71) Applicants: Karl Erich Jaeger, Mulheim (DE); Thomas Drepper, Stolberg (DE); Stephan Endres, Dusseldorf (DE); Janko Potzkei, Dusseldorf (DE)

(72) Inventors: Karl Erich Jaeger, Mulheim (DE); Thomas Drepper, Stolberg (DE); Stephan Endres, Dusseldorf (DE); Janko Potzkei, Dusseldorf (DE)

(73) Assignee: EVOCATAL GMBH, Düsseldorf (DE)

(21) Appl. No.: 13/817,750

(22) PCT Filed: Aug. 16, 2011
(86) PCT No.: PCT/EP11/64058

§ 371 Date: May 21, 2013

(30) Foreign Application Priority Data
Aug. 16, 2010 (DE) 102010036997.7

(51) Int. Cl. C12N 13/00; C07K 14/21; C07K 14/32

(52) U.S. Cl. CPC C12N 13/00; C07K 14/32; C07K 14/21
USPC 435/69.1; 530/350; 435/471

(57) ABSTRACT

The present invention relates to the use of a protein comprising an LOV domain for the photosensitive defunctionalization of a molecule and to a method for the photosensitive defunctionalization of a target molecule.

### REFERENCE TO RELATED APPLICATIONS

This application is a U.S. national stage application of International Patent Application No. PCT/EP2011/064058, filed Aug. 16, 2011, and claims the priority benefit of German Application No. 102010036997.7, filed Aug. 16, 2010, the entire disclosures of which are incorporated herein by reference.

### FIELD OF THE INVENTION

The present invention relates to the use of a protein comprising a LOV domain for photosensitive defunctionalization and to a method for photosensitive defunctionalization.

### BACKGROUND OF THE INVENTION

Photosensitizers are chromophores or chromophore-binding proteins which absorb light and then transfer the energy non-radiatively to the atoms or molecules to be caused to react. This occurs because the photosensitizer cannot give up its energy to the surrounding medium by emitting light or by non-radiative pathways. Such photosensitizers which generate reactive oxygen species (ROS) when irradiated with light have attracted a great deal of attention. They transfer the “surplus” energy of the chromophore excited by the light either in the form of electrons to elemental oxygen, whereupon superoxide anions (O₂.⁻) are formed, or the chromophore changes into the triplet state. From this relatively stable state, energy is transferred to elemental oxygen in the form of electrons, whereupon radicals in the form of singlet oxygen (¹O₂) are formed (Jacobson et al., 2008). ROSs can oxidize the side chains of various amino acids, whereupon both intramolecular and protein-protein cross-linking occurs, which culminates in protein aggregation and/or the loss of enzymatic activity.

By using photosensitizers, prokaryotic and eukaryotic cells which express them or have taken them up can be killed off simply by irradiating them with light. Principally, this is due to the increased intracellular production of reactive oxygen species causing defects in cellular components (protein cross-linking, protein aggregation and loss of activity), which in turn results in cell death.

In addition, the toxic effect of photosensitizers can be spatially and temporally limited by using the CALI-technique (chromophore-assisted light inactivation), whereby a specific inactivation of individual proteins and/or cell structures is made possible. In this technique, the photosensitizer is conjugated with an antibody or expressed in vivo in a defined cell compartment or fused with a target protein. The region of the cell to be investigated is specifically irradiated with light using a laser, which light is absorbed by the photosensitizer whereupon, for example, increased generation of reactive oxygen species (ROS) occurs. Since these radicals are rather short-lived, their radius of toxicity is greatly limited and so, for example, deliberate inactivation of the fused target protein is made possible. Thus, this technique constitutes a multi-faceted molecular tool for specific loss of function analyses (Jacobson et al., 2008).

In the current literature, various fluorescence proteins have been described which have a phototoxic effect on bacterial cells which is induced by light. These proteins are representatives of the eGFP (enhanced green fluorescent protein) class, or homologous proteins thereof such as KillerRed a derivative of the chromoprotein anm2CP²⁰ from Anthomedusae spec. This fluorescence protein has a maximum fluorescence emission at 610 nm and the excitation maximum is 585 nm. Studies have established that the strength of the phototoxicity of KillerRed is dependent on the type (wavelength) of the light used. In this regard, it has been shown that the strongest killing effect can be reached with green light (540-580 nm). Furthermore, a toxic effect of the photosensitizer could be obtained on eukaryotic cell lines using human kidney cells (40-60% killed after 10 minutes irradiation with green light).

In contrast to current photosensitizers which, for use in living cells, have to be taken in by means of an active transport mechanism or permeabilization of the cell membrane, genetically coded fluorescence proteins such as KillerRed have the advantage that they can be used non-invasively. In addition, fluorescence proteins can be precisely localized in desired cell compartments by fusion with suitable signal sequences, or they can be fused to any target proteins.

Until now, KillerRed as well as some derivatives of GFPs have been the only known genetically encoded photosensitizers which can be used both for the CALI method and also which can exert a toxic effect on cells.

### SUMMARY OF THE INVENTION

Thus, it would be highly advantageous if further photosensitizers of this type were available.

Thus, the aim of the present invention is to provide other genetically encoded photosensitizers which can be used for photosensitive defunctionalization of a molecule.

The present invention also aims to provide a method for the photosensitive defunctionalization of a target molecule.

Thus, the present invention concerns the use of a fluorescence protein comprising a LOV domain for the photosensitive defunctionalization of a molecule, wherein at least one cysteine in the LOV domain is replaced by another amino acid which does not covalently bind any FMN.

### BRIEF DESCRIPTION OF THE DRAWINGS

FIG. 1 shows a diagrammatic representation of reactive oxygen species production by the excited photosensitizer;

FIG. 2 shows the phototoxic effect of FbFP with SEQ ID No. 6 on E. coli cells;

FIG. 3 shows that the phototoxic effect of FbFP with SEQ ID No. 6 can be produced exclusively by blue light;

FIG. 4 shows that the phototoxic effect of FbFP with SEQ ID No. 6 can be produced exclusively by blue light;

FIG. 5 shows the temporal profile of the phototoxic effect of FbFP with SEQ ID No. 6;

FIG. 6 shows the diagrammatic representation of the fusion protein obtained from FbFP with SEQ ID No. 6 and YFP;

FIG. 7 and FIG. 8 show a cloning strategy for the production of the fusion protein from FIG. 6;

FIG. 9 and FIG. 10 show a cloning strategy for the production of the fusion protein from FIG. 6;

FIG. 11 shows the CALI inactivation of YFP.

### DETAILED DESCRIPTION OF PREFERRED EMBODIMENTS

The term “protein” as used below should be understood to mean macromolecules built up from amino acids. Recombinant proteins and special proteins, for example antibodies and enzymes, also fall within this definition.

The term “fluorescence protein comprising a LOV domain” as used below should be understood to mean a protein that comprises a light, oxygen or voltage (LOV) domain, in which at least one cysteine is replaced by another amino acid which does not covalently bind any FMN.

Preferably, the at least one cysteine is replaced by alanine

Preferably again, the LOV domain comprises one of SEQ ID No. 1-4 (see Table 2).

The term “photosensitive defunctionalization” as used below should be understood to mean a procedure in which the fluorescence protein comprising a LOV domain absorbs light of a specific wavelength and then transfers this energy non-radiatively onto a molecule which is to be caused to react. In this regard, the term “defunctionalization” should be understood to mean “causing a specific reaction” such as, for example, chromophore assisted light inactivation (CALI) and/or a phototoxic reaction.

The term “phototoxic reaction” as used below should be understood to mean that the photosensitive defunctionalization damages a cell in such a way that growth is instigated and/or it dies off.

The term “molecule” as used below should be understood to mean an element which has its mode of action changed by the photosensitive defunctionalization. As an example, the molecule may be an antibody or an enzyme. In one of these, the photosensitive defunctionalization can cause a structural change of the type that renders it inactive. If the molecule is a cellular component, for example a protease or part of a protease, the photosensitive defunctionalization may cause defects which again, for example, cause protein cross-linking or protein aggregation and a concomitant loss of activity which can ultimately result in cell death.

Recently, a completely novel fluorescence protein family comprising LOV domains or recombinant variations of bacterial blue light receptors of the LOV family has been developed. In contrast to fluorescence proteins like GFP, the novel fluorescence markers are very small (16-19 kDa). In order to increase the FMN-dependent fluorescence of the blue light receptors and thus to allow them to be used as fluorescence markers, the bacterial proteins were changed using up-to-date procedures known as directed evolution. By means of such mutations, auto-fluorescence of the proteins was drastically increased, whereupon FMN-binding fluorescence proteins (FbFPs) were produced. The photochemical characterization of the novel marker proteins showed that the FbFPs emit a blue-green fluorescence (495 nm) after excitation with blue light (450 nm). The novel marker proteins could be expressed in various prokaryotic and eukaryotic host cells and the characteristic fluorescence of the FbFP could be assayed in vivo.

Examples of proteins comprising a LOV domain are the blue light receptor YtvA from B. subtilis and the sensory box protein SB2 from Pseudomonas putida.

The blue light receptor YtvA is a 261 amino acid protein which was classified as a putative protein kinase during the complete genome sequencing of B. subtilis. It plays a role as a positive regulator in the σ^{B}-mediated stress response of B. subtilis. YtvA could be identified, by means of sequence homology comparisons, with the primary structure of plant blue light receptors, phototropins. YtvA consists of an N-terminal LOV domain and a C-terminal STAS (sulfate transporter antisigma) domain. The LOV domain exhibits a special folding motif formed by a 5-stranded anti-parallel β-sheet with sides flanked by α-helices. The LOV domain contains the known consensus sequence NCRFLQG (SEQ ID No. 7) from plant phototropins, wherein the photoactive cysteine contained in it covalently binds the FMN cofactor as a chromophore during the LOV-specific photocycle. In this regard, excitation with light at a wavelength of 450 nm causes the YtvA to change over from the ground state into a photoproduct which absorbs at 383 nm and has an emission maximum of 498 nm. Within a short time period (approx. 1.6 μs), this photoproduct decays to a photoadduct in which the FMN is covalently bound. In this form, the LOV domain loses its property of fluorescence until the ground state is regained.

By using sequence homology comparisons of the LOV domain of YtvA from B. subtilis with the genome sequence of the gram-negative rod bacterium P. putida KT2440, a gene was identified therein which encodes a putative LOV protein. It is the 151 amino acid SB2 (sensory box 2), which contains only one LOV domain. The SB2 protein has the characteristic consensus sequence of LOV domains (SEQ ID No. 7).

Surprisingly, it is possible to carry out a specific reaction of a molecule by photosensitive defunctionalization using the fluorescence protein of the invention.

The use of such fluorescence proteins can, for example, be envisaged in the biotechnology and biomedical fields. Thus, for example, in photodynamic cancer therapy, LOV domains of fused protein-antibody-proteins can tag cancer cells in a precise manner and successfully kill them by irradiation with light. Furthermore, process-relevant reactions in bacterial and eukaryotic host organisms (such as any biotechnological processes, including bacteria-based cancer therapies), can be stopped within a short period by irradiation with light.

In this manner, in this unforeseen way, it has been shown for the first time that a protein which is not related to GFP can be used for photosensitive defunctionalization of a molecule.

In addition, the use of fluorescence proteins comprising a LOV domain from the families described above can for the first time use fluorescence reporters to kill off specific cells simply by irradiating with blue light. In contrast to KillerRed, which is already known, which produces more oxygen radicals upon irradiation with green light (λ=540-580 nm), a completely novel way of bringing about the photosensitive defunctionalization of a molecule is presented.

Consequently, a further advantage of this fluorescence protein comprising a LOV domain is that it can be used in combination with KillerRed, so that irradiation with light of various wavelengths produces different defunctionalizations.

Clearly, it is also possible for the protein of the invention to comprise more than one LOV domain.

In a further embodiment of the invention, the protein is used in a solution or a cell.

The term “cell” as used below should be understood to mean both prokaryotic and eukaryotic cells. The cells do not necessarily have to be living cells at the time a protein comprising a LOV domain is used for the photosensitive defunctionalization of a molecule.

The term “solution” as used below should be understood to mean a liquid. It may be a homogeneous mixture or a suspension.

Thus, the protein comprising a LOV domain for photosensitive defunctionalization may be used in a solution, for example in order to examine an interaction between proteins or the effect of one protein on other proteins.

Alternatively, the protein can be used in a cell; here again, an interaction between proteins or the effect of one protein on other proteins may be examined, for example.

In both cases, the fluorescence protein in accordance with the invention can, for example, have a direct influence on a target protein so that, following photosensitive defunctionalization, this effect no longer takes place. However, equally, the fluorescence protein of the invention may be envisaged as acting indirectly on the target protein; for example, it might bind the binding partner of the target protein. In this manner, it may be possible that, following photosensitive defunctionalization by the fluorescence protein of the invention, the binding partner of the target protein would no longer be capable of interacting with the target protein since its structure has been changed.

Similarly, it is also conceivable that, rather than an interaction or reaction between two proteins, the interaction or reaction of a nucleic acid with at least one protein or the interaction or reaction between nucleic acids will be examined.

Further, the use of the protein comprising a LOV domain for photosensitive defunctionalization in a cell may aid in elucidating the function of specific proteins in specific cell compartments, to localize specific proteins and/or to comprehend cell processes.

By fusion of the protein comprising a LOV domain with another protein, it is possible, for example, to obtain specific inhibition of a protein and cell function (for example a specific metabolic pathway) by photosensitive defunctionalization.

In a further embodiment of the invention, the photosensitive defunctionalization concerns chromophore assisted light inactivation (CALI) and/or a phototoxic reaction.

The term “phototoxic reaction” as used below should be understood to mean that the photosensitive defunctionalization damages a cell in such a manner that it alters its growth and/or kills it.

Thus, by using the protein comprising a LOV domain for photosensitive defunctionalization, it is possible to interrupt the growth of target cells precisely at a freely selectable time, without affecting other cells which might be in the vicinity. As an example, feeder cells could be specifically killed off when they are no longer required.

Furthermore, using the CALI technique via a spatially and temporally limited light irradiation means that the photosensitive defunctionalization of a molecule is limited spatially and temporally, whereupon a precise inactivation of individual proteins and/or cell structures is made possible. It has already been shown that CALI-mediated intracellular processes in living cells can be influenced. Thus, for example, signal transduction pathways or protein interactions can be manipulated in living cells.

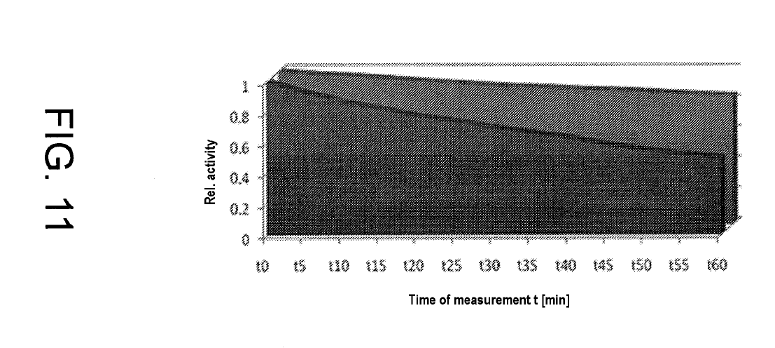

An example of a molecule which has been defunctionalized by means of CALI using the protein of the invention having a LOV domain is YFP, the fluorescence of which decays rapidly after the photosensitive defunctionalization (see FIG. 11).

Preferably, the protein with a LOV domain of the invention is photoactive at wavelengths of 380-490 nm; photosensitive defunctionalization is thus carried out at these wavelengths.

In a further embodiment, in the LOV domain, more precisely, the cysteine in position 53 of SEQ ID No. 6 is replaced by another amino acid which does not covalently bind FMN.

This produces a phototoxic effect under the influence of light. For other proteins which have the same sequence, but wherein the cysteine in position 53 is present, no phototoxic effect could be discerned.

In a further embodiment, said LOV domain is characterized in that
a) it is encoded by the nucleic acids of SEQ ID No. 5 or a fragment, a variant, a homologue or a derivative of this sequence,
b) it is encoded by a nucleic acid which can hybridize with the nucleic acids from a) under stringent conditions,
c) it is encoded by a nucleic acid which has at least 70%, preferably 95% identity with one of the nucleic acids from a) or b),
d) it is encoded by a nucleic acid which can hybridize under stringent conditions with the complementary nucleic acid of one of the nucleic acids from a)-c),
e) it is encoded by a nucleic acid which, compared with the nucleic acids from a)-d), has at least one silent mutation of a single nucleotide (as allowed by the degeneration of the genetic code),
f) it is encoded by a nucleic acid the code for which has been optimized for a specific expression system compared with the nucleic acids from a)-e),
g) it comprises an amino acid sequence in accordance with SEQ ID No. 6 or a fragment, a variant, a homologue or a derivative of this sequence,
h) it comprises an amino acid sequence which has a sequence identity of at least 70%, preferably 95% with the amino acid sequences from g).

The term “nucleic acid” as used below should be understood to mean a single or double-stranded macromolecule which is formed from nucleotides. The most common nucleic acids are deoxyribonucleic acid (DNA) or complementary DNA (cDNA) and ribobucleic acid (RNA). DNA contains the nucleobases adenine, cytosine, guanine and thymine, the latter being specific to DNA. RNA contains the same nucleobases or nucleotides, except that thymine is replaced by uracil.

Examples of synthetic nucleic acids are peptide nucleic acid (PNA), morpholino and locked nucleic acid (LNA), as well as glycol nucleic acid (GNA) and threose nucleic acid (TNA). The construction of the backbone of each of these nucleic acids differs from nucleic acids of natural origin.

The term “complementary” as used below should be understood to mean the nucleic acids which are complementary to the used/discussed nucleic acids. This is an important concept in molecular biology, since it concerns an important property of double-stranded nucleic acids such as DNA, RNA or DNA:RNA duplexes. One strand is complementary to the other in that the base pairs of both strands are bonded non-covalently via two or three hydrogen bonds. In principle—there are exceptions for thymine/uracil and the wobble complex of tRNA—there is only one complementary base for each base of a nucleic acid. Thus, it is possible to reconstruct the complementary strand of a given individual strand. This is essential to DNA replication, for example. As an example, the complementary strand for the DNA sequence

a. 5′ A G T C A T G 3′
b. 3′ T C A G T A C 5′

is

In the case of DNA, the term “complementary” can also denote cDNA. cDNA is synthesized using the reverse transcriptase enzyme from RNA, for example mRNA.

The term “hybridize” or “hybridization” as used below should be understood to mean the procedure whereby a nucleic acid becomes bonded to a more or less completely complementary nucleic acid with the formation of hydrogen bonds between the respective complementary nucleobases.

The term “hybridize under stringent conditions” as used below should be understood to mean that the conditions for the hybridization reaction are set such that only completely complementary bases can form hydrogen bonds. The stringency may be influenced by the temperature, for example.

The term “silent mutation” as used below should be understood to mean the phenomenon whereby a mutation in a section of a nucleotide acid does not result in any consequences. In such a case, the information content of the gene is not changed, because an amino acid chain is encoded by different groups of three successive nucleobases—known as triplets or codons.

The term “fragment” as used below should be understood to denote a portion of a nucleic acid or an amino acid sequence wherein some parts are missing a given nucleic acid or an amino acid sequence, but wherein at least a part of its activity, for example as regards fluorescence properties, enzyme activity, or binding to other molecules, is retained.

The term “variant” as used below should be understood to mean a nucleic acid or an amino acid sequence which has a structure and biological activity which is essentially the same as the structure and biological activity of a specific nucleic acid or an amino acid sequence.

The term “derivative” as used below should be understood to mean a related nucleic acid or amino acid sequence which has similar characteristics with respect to a target molecule as a given nucleic acid or amino acid sequence.

The term “homologue” as used below should be understood to mean a nucleic acid or an amino acid sequence the sequence of which has at least one nucleotide or an amino acid which has been added, deleted, substituted or modified in another manner compared with the sequence of a given nucleic acid or amino acid sequence. However, the homologue must have essentially the same properties as the given nucleic acid or amino acid sequence.

The term “optimized for a specific expression system” as used below should be understood to mean that a cDNA is matched to the codon usage of the organism in which it is expressed. The codon usage, or codon bias, describes the phenomenon that variants of the universal genetic code are often used in different ways by different species.

The term “sequence identity of at least X %” as used below should be understood to mean a sequence identity determined by sequence alignment using a BLAST algorithm available on the homepage of the NCBI.

In this embodiment, cysteine is replaced by alanine, as has occurred in SEQ ID No. 5 or respectively SEQ ID No. 6.

The phototoxicity of the fluorescence protein with SEQ ID No. 6, termed FbFP with SEQ ID No. 6, is most probably due to the increased formation of oxygen radicals, as has been shown for the fluorescence protein KillerRed.

The LOV consensus sequence after this exchange is thus no longer NCRFLQ (SEQ ID No. 7), but NXRFLQ.

The term “consensus sequence” as used below should be understood to mean an amino acid sequence which is in agreement with the LOV domains of YtvA from B. subtilis and SB2 from P. putida.

In contrast to the well-known and much-described photosensitizer KillerRed, the chromophore of which can only be excited by green light, FbFP with SEQ ID No. 6 specifically absorbs blue light, and thus broadens the palette of genetically coded photosensitizers. The development of FbFP with SEQ ID No. 6 means that for the first time, combined applications with KillerRed are possible in order to allow precise phototoxic processes to be carried out with two colours of light.

The present invention also concerns a method for the photosensitive defunctionalization of a target molecule, comprising at least the following steps:
introducing a vector which encodes a protein which is photoactive at wavelengths of 380-490 nm into a cell which contains the target molecule, and expressing the protein in this cell or coupling a protein which is photoactive at wavelengths of 380-490 nm to a target molecule
irradiating the cell or the protein—target molecule complex with light with wavelengths of 380-490 nm.

The term “target molecule” as used below should be understood to mean an element which is changed in its mode of action by the photosensitive defunctionalization. The terms “molecule” and “target molecule” are thus synonymous, but the term “target molecule” has been selected in order to make the claim easier to construe.

The term “coupling” as used below should be understood to mean that the protein is brought into the spatial vicinity of the target molecule for photosensitive defunctionalization so that a protein-target molecule complex can form. This can occur in a variety of ways, for example by covalent and non-covalent binding, such as in biotin-streptavidin binding.

Irrespective of whether a target is to be photosensitively defunctionalized in a cell or in solution, the vector is introduced into the cell and the protein for the photosensitive defunctionalization is expressed therein, or the protein for photosensitive defunctionalization is coupled to a target molecule if the photosensitive defunctionalization is to take place in solution.

Surprisingly, it has been shown that a photosensitive defunctionalization can also be carried out with blue light with a wavelength of 380-490 nm.

In one embodiment, the method comprises the following steps before coupling the protein to the target molecule:
introducing a vector into a cell, which vector encodes the protein which is photoactive at wavelengths of 380-490 nm,
expressing the protein in the cell
extracting the protein.

The term “extracting” the protein should be understood to mean all steps of the method which are necessary following expression of the protein so that subsequently, it can be used outside the cell. Thus, for example, “extracting” includes cell digestion, secreting of the protein by the cell itself, any purification required, and any concentration.

These steps of the method are preferably then carried out when the protein for the photosensitive defunctionalization is to be used for coupling to a protein outside a cell.

In a preferred embodiment, the method additionally comprises the step in which the protein-target molecule complex is introduced into a cell prior to irradiation.

This step of the method is thus preferably carried out when the protein for the photosensitive defunctionalization is not expressed in the cell in which it is to be subsequently used for photosensitive defunctionalization.

Thus, for example, the protein for photosensitive defunctionalization could be coupled to a target molecule and the two proteins may be introduced into a cell together, for example by means of protein transduction using a protein transduction domain.

In a further embodiment of the method, the protein and the target molecule can be expressed together as a transcription unit.

This embodiment is preferred, for example, if the function of the target molecule in the cell is to be examined, since joint expression of the target molecule with the protein for the photosensitive defunctionalization ensures co-localization—for example by fusion of the two elements.

In a further embodiment, the cell which expresses the protein is preferably a bacterium selected from the group consisting of Escherichia coli, Rhodobacter capsulatus, Pseudomonas putida and/or Bacillus subtilis.

As an example, the protein for the photosensitive defunctionalization, which is photoactive at wavelengths of 380-490 nm, could be introduced into and expressed in a bacterial cell, for example E. coli, by means of a vector. In this manner, the E. coli cells are either lysed, in order to obtain the protein for the photosensitive defunctionalization, or the vector also contains a sequence which allows the protein for photosensitive defunctionalization to be released into the surrounding medium.

Further advantages and advantageous embodiments of the method of the invention will become apparent from the figures and exemplary examples and from the following description. It should be noted that the figures and exemplary examples are purely descriptive in nature and should not be considered to limit the invention to any specific form.

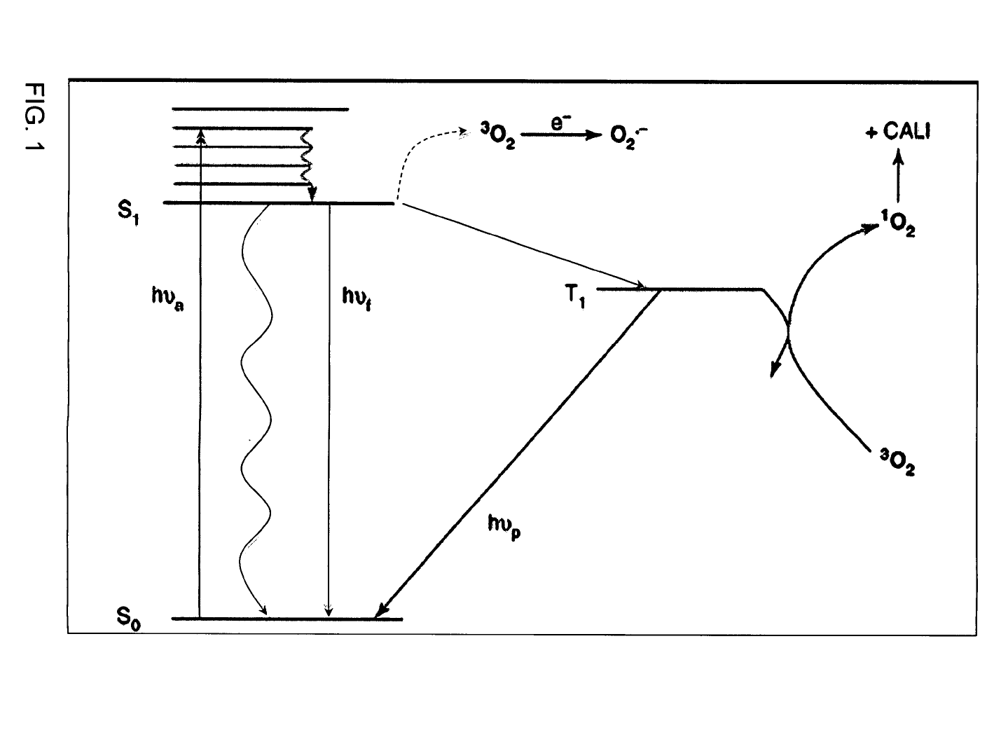

FIG. 1 shows a diagrammatic representation of reactive oxygen species production by the excited photosensitizer. The absorption of a photon (hνₐ) leads to excitation of the chromophore into the lowest excited singlet state (S₁). The energy from this state can either be transferred non-radiatively to the surrounding medium (wavy arrow), or by the emission of light (hν_{f}) or by electron transfer to elemental oxygen, to produce a superoxide anion. In addition, the excited chromophore can decay into the triplet state (T₁), from which the energy is released by phosphorescence (hνₚ) or is transferred by electron transfer to elemental oxygen. In this process, singlet oxygen (¹O₂) is generated (figure modified from Jacobson et al., 2008).

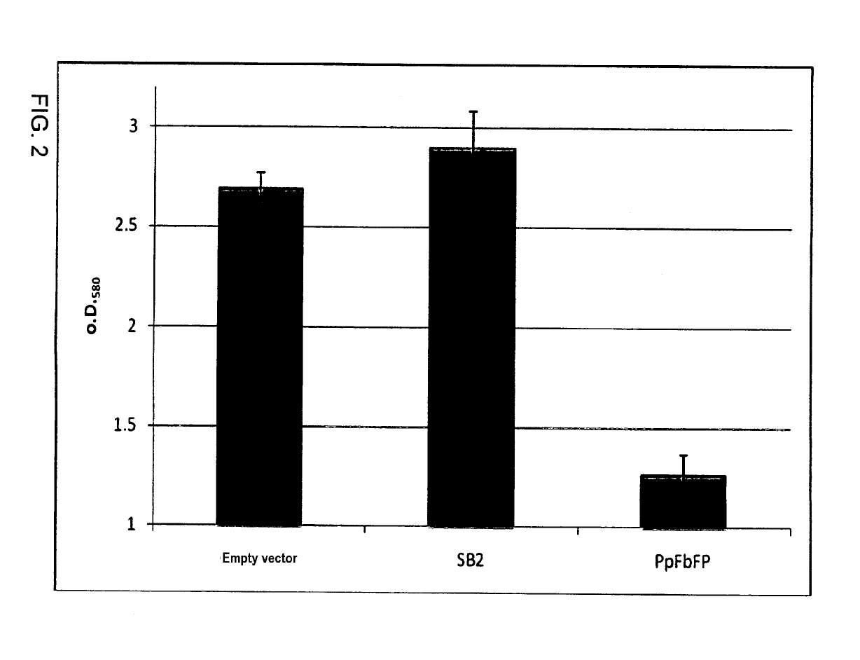

FIG. 2 shows the phototoxic effect of FbFP with SEQ ID No. 6 (PpFbFP) on E. coli cells. It shows the OD₅₈₀ of FbFP with SEQ ID No. 6-expressing E. coli DH5α cultures 24 hours after inoculation, wherein the cultures were irradiated with blue light (λ=460 nm)2 hours after inoculation for a period of 2½ hours. The values shown are the means of three different, independent measurements; the error bars are the standard deviations of these measurements.

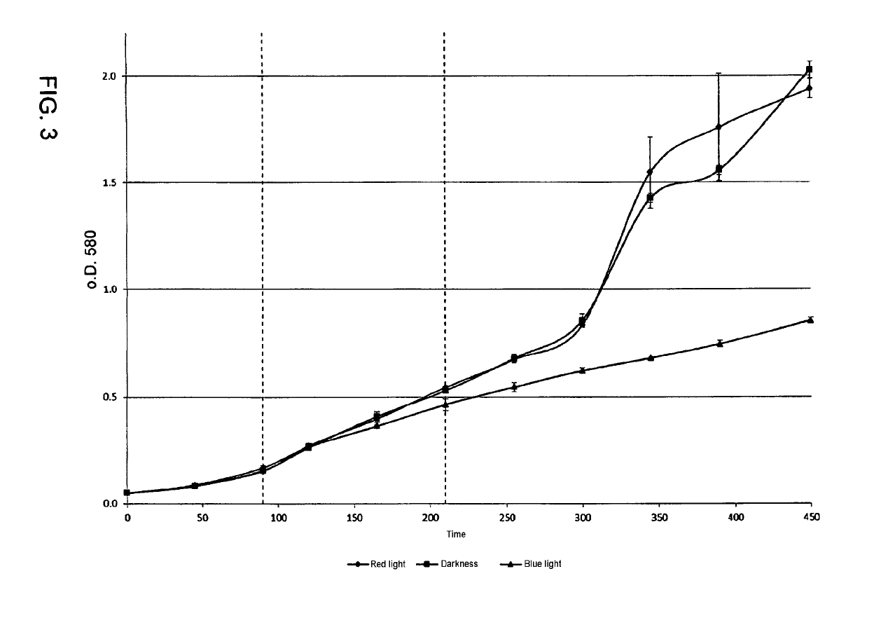

FIG. 3 shows that the phototoxic effect of FbFP with SEQ ID No. 6 is exclusively obtained with blue light. It shows the optical density (at 580 nm) of FbFP with SEQ ID No. 6-expressing, transformed E. coli DH5α strains as a function of time. The cultures were inoculated with a quantity of cells corresponding to an OD₅₈₀=0.05 and incubated at 37° C. on an incubator shaker in the dark. 90 minutes after inoculation, the cultures underwent either direct blue light irradiation (λ=460 nm, triangular symbols) or direct red light irradiation (λ=856 nm, diamonds) for a period of 2 hours. The control was a culture left in the dark (squares). The values shown each represent the mean of three different, independent measurements; the error bars are the standard deviations of these measurements.

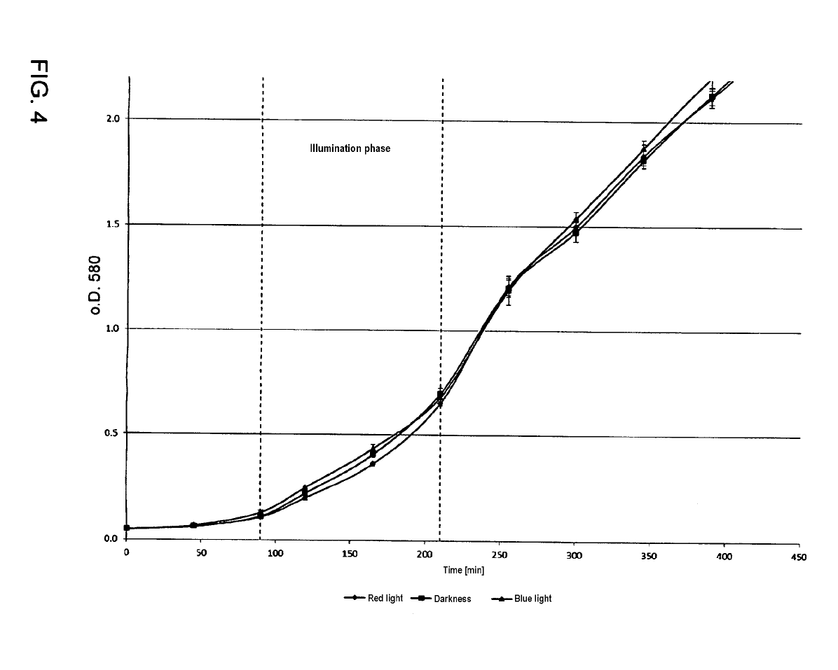

FIG. 4 shows that the phototoxic effect of FbFP with SEQ ID No. 6 is exclusively obtained with blue light. It shows the change in optical density with time (at 580 nm) of E. coli DH5α strains transformed with the pRhokHi-2 empty vector. The cultures were inoculated with a quantity of cells corresponding to an OD₅₈₀=0.05 and incubated at 37° C. on an incubator shaker in the dark. 90 minutes after inoculation, the cultures underwent either direct blue light irradiation (λ=460 nm, triangular symbols) or direct red light irradiation (λ=856 nm, diamonds) for a period of 2 hours. The control was a culture left in the dark (squares). The values shown each represent the mean of three different, independent measurements; the error bars are the standard deviations of these measurements.

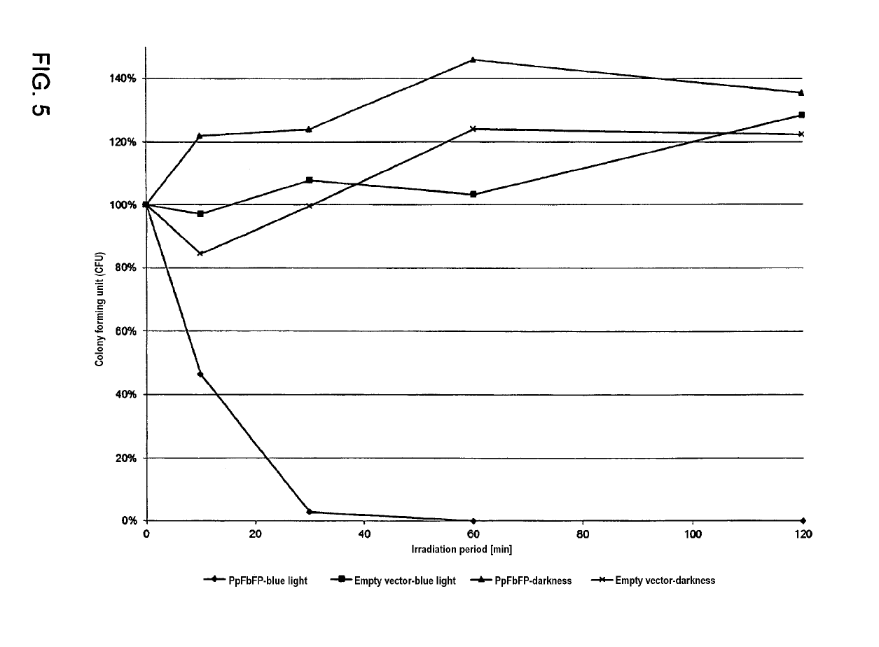

FIG. 5 shows the plot against time of the phototoxic effect of FbFP with SEQ ID No. 6. It shows the change with time of the colony forming units of the FbFP with SEQ ID No. 6-expressing culture (lines with diamonds and triangles), or the empty vector control (lines with squares and crosses) as a function of the duration of irradiation. The relative values were determined by taking cell samples (OD₅₈₀=0.1) at the given points in time, which were plated out in different dilutions onto LB solid medium. The count of grown colonies was determined, factoring in the respective dilution factor. Next, the ratio of the respective sample to the corresponding original sample (0 minutes) was calculated. The values shown correspond to the mean of three independent measurements.

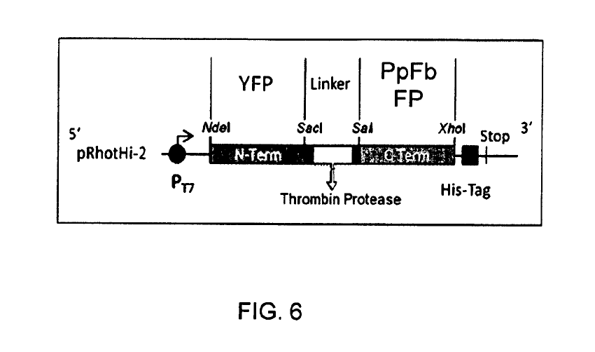

FIG. 6 shows the diagrammatic representation of the FbFP with SEQ ID No. 6—YFP fusion proteins produced.

FbFP with SEQ ID No. 6 (PpFbFP) was fused to the yellow fluorescing protein YFP (SEQ ID No. 9). The proteins were bound by means of a linker (SEQ ID No. 10 and 11), which contains a “multiple cloning site” (MCS) with the cleavage sites for the restriction endonucleases KpnI, NdeI, BamHI, SacI, SalI, HindIII, XhoI and Cfr42I. In order to express the fusion protein, the expression vector pRhotHi-2 was selected. In addition, the fusion protein was provided with a His₆ tag, by means of which the recombinant protein could be readily purified by affinity chromatographic methods.

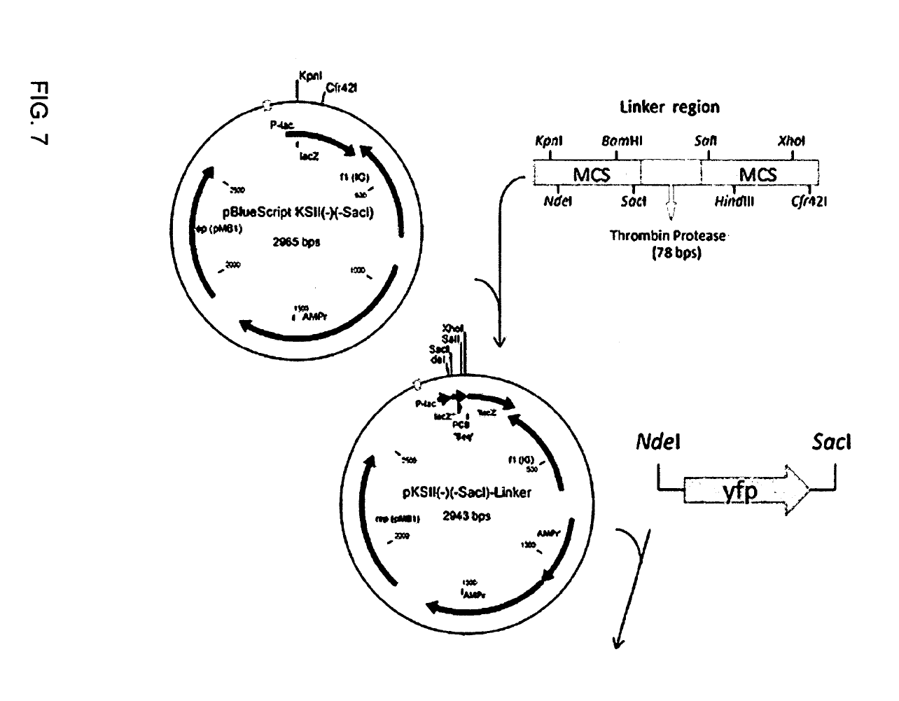

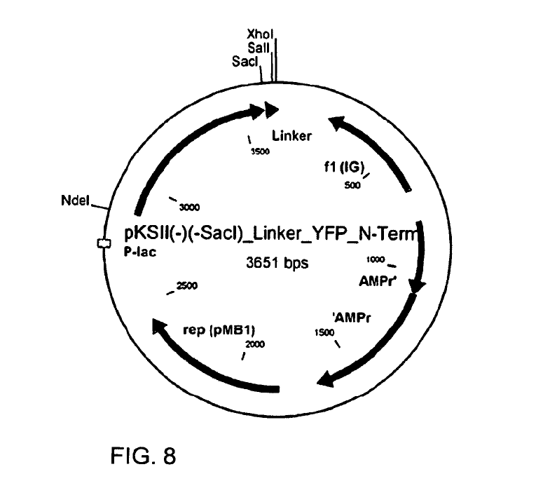

FIG. 7 and FIG. 8 show a cloning strategy for the production of the fusion protein of FIG. 6.

The photosensitizer gene for FbFP with SEQ ID No. 6 (SEQ ID No. 5), the target gene YFP (SEQ ID No. 8) and the linker (SEQ ID No. 10 and 11) were cloned into the cloning vector pBlueScript KSII(−), which allows for blue-white selection in E. coli DH5α cells.

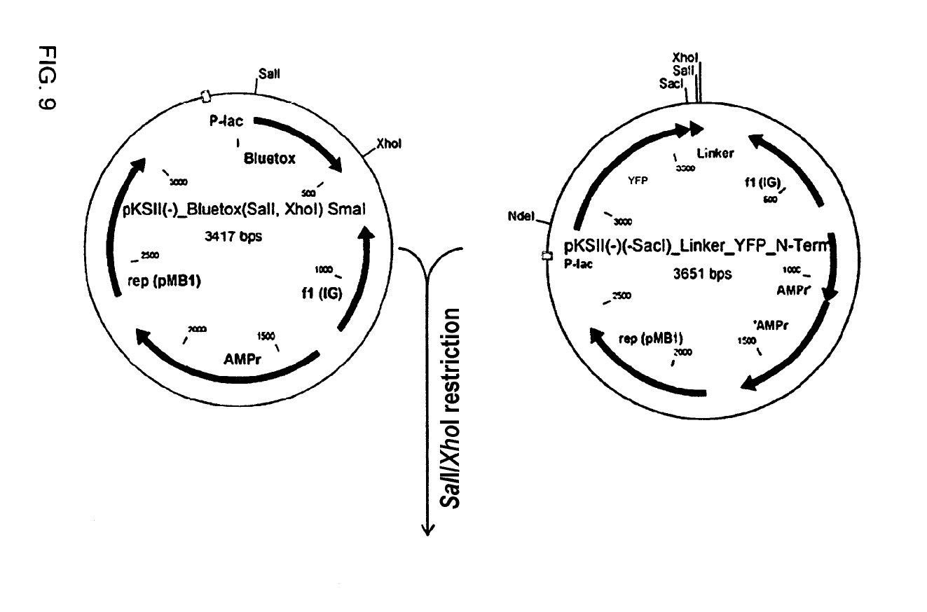

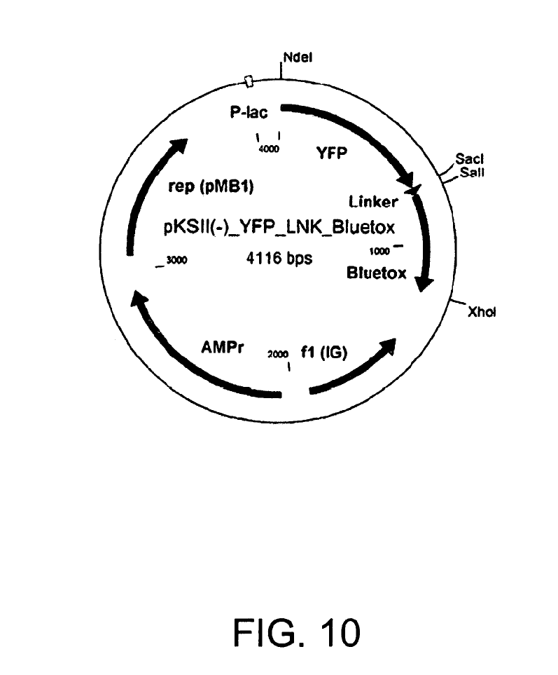

FIG. 9 and FIG. 10 show a cloning strategy for the production of the fusion protein.

The map indicated shows the final fusion of the C-terminal fusion of the FbFP with SEQ ID No. 6 with YFP (SEQ ID No. 9) by restriction digestion with the restriction endonucleases SalI/XhoI and subsequent ligation in pBlueScript KSII(−).

FIG. 11 shows the CALI inactivation of YFP.

It shows how blue light irradiation of the fusion protein of FIG. 6 (dark grey) leads to a rapid decay of YFP fluorescence. The YFP protein (SEQ ID No. 9) is thus inactivated by the CALI method, dependent on FbFP with SEQ ID No. 6. In contrast, blue light irradiation has no effect on the activity of the non-fused YFP protein (pale grey).

### Exemplary Embodiments

Results will be presented below which show that surprisingly, the LOV fluorescence protein FbFP with SEQ ID No. 6 can be used as a blue light-induced photosensitizer. The phototoxic effect of FbFP with SEQ ID No. 6 has been demonstrated with the aid of the Gram-negative bacterium E. coli DH5α. The expression system used was the pRhokHi-2 vector which allows constitutive expression of all LOV proteins through the aphII promoter.

1. Influence of Blue Light Irradiation on the Growth of E. coli Cultures which Express FbFP with SEQ ID No. 6.

In order to demonstrate the phototoxic influence of blue light irradiation on growing E. coli cultures which express FbFP with SEQ ID No. 6, initially, the LOV fluorescence protein FbFP with SEQ ID No. 6 and its corresponding wild type protein SB2 were expressed using the pRhokHi-2 expression vector. To this end, the expression constructs pRhokHi-2_PpFbFP and pRhokHi-2_SB2 were initially cloned. The genes were amplified by PCR with the aid of specific primers (SEQ ID No. 12 & 13) and simultaneously, the 5′ end was provided with the NdeI and the 3′ end was provided with the XhoI restriction endonuclease-specific recognition sequences. Next, the PCR products were cloned by means of NdeI/XhoI double restriction digestion into the expression vector, which had also been hydrolysed using the cited endonucleases. Successful cloning was checked by means of an appropriate test restriction and verified by sequencing the cloned DNA fragments.

Initially, in order to positively demonstrate the selective blue light-dependent phototoxicity of the FbFP with SEQ ID No. 6, the expression constructs which were produced as well as the pRhokHi-2 empty vector (negative control) were transformed in the bacterial strain E. coli DH5α and multiplied on LB agar plates under selection pressure. One colony from the grown cultures was transferred into a 5 mL LB full medium culture and then incubated overnight under selection pressure at 37° C. Next, 50 mL of LB full medium cultures from these pre-cultures was inoculated to a concentration of OD₅₈₀=0.05 and then incubated under selection pressure on an incubator shaker at 37° C. After incubating for 2 hours, the cultures were subjected to direct blue light irradiation (λ=460 nm), which was switched off again after an irradiation period of 2½ hours. The phototoxic effect of the LOV proteins was then determined with the aid of the cell density obtained in the batch cultures after 24 hours cultivation.

It will be observed from the determined cell densities that the expression of SB2 under blue light irradiation has no negative effect on the growth of E. coli cultures, since the cell density of the corresponding strain and that of the negative control were the same. Surprisingly, however, the cell density was significantly lower for those cultures which express the fluorescence protein FbFP with SEQ ID No. 6. Apart from this culture, which only grew to an OD₅₈₀ of approximately 1.3, the cell density of all of the other cultures at the same point in time was between 2.6 and 2.8. Thus, when irradiated, FbFP with SEQ ID No. 6 has a generally growth-inhibiting or growth-reducing effect on E. coli cells.

2. Influence of Irradiation with Light of Different Wavelengths on the Growth of E. coli Cultures which Express FbFP with SEQ ID No. 6.

In order to provide a better indication of the light-dependent phototoxic effect of FbFP with SEQ ID No. 6, the protein was examined afresh in a similarly designed growth experiment. This was to show that the phototoxic effect of FbFP with SEQ ID No. 6 is exclusively brought about by light which can be absorbed by the FMN chromophore. To this end, the appropriate E. coli cultures were additionally irradiated with red light (λ=856 nm). The negative control was exactly as described in the preceding experiment, namely an E. coli culture transformed with the pRhokHi-2 empty vector.

One colony from each of the strains expressing FbFP with SEQ ID No. 6 was transferred into a 5 mL LB full medium pre-culture and incubated overnight under selection pressure at 37° C. on a rotary shaker. Next, 50 mL of LB test culture from this pre-culture, to which 50 μg/mL of kanamycin had been added, was inoculated to a cell count giving an OD₅₈₀ of 0.05 and then incubated at 37° C. in the dark on an incubator shaker. This time, the cultures underwent light irradiation for a time period of 2 hours, starting 90 minutes after inoculation. Beginning the irradiation at an earlier point in time was intended to minimize a possible self-shading effect of the cells in the cultures. The growth profile of the cultures was then recorded at regular intervals by photometric turbidity measurements.

The growth curves of the cultures expressing FbFP with SEQ ID No. 6 confirmed the results of the preceding experiment. This experiment also showed up the influence of blue light on cell growth. Thus, a “kink” in the growth curve can be seen approximately half an hour after the start of the blue light illumination. This effect was not observed with the cultures grown under red light irradiation or in the dark. Thus, it was clear that the negative influence on growth is specifically induced by blue light. In addition, after 450 minutes, the cultures grown under red light or in the dark reached a cell density which had an OD₅₈₀ of approximately 2, and thus was more than double that of the cell density of the cultures grown under blue light irradiation.

3. Proof of Phototoxic Effect of FbFP with SEQ ID No. 6.

In order to be able to demonstrate that the growth inhibition of FbFP with SEQ ID No. 6 under blue light irradiation is actually a phototoxic effect and not a bacteriostatic effect, during continued growth, respectively immediately before the start of the blue light irradiation and after an illumination period of 90 minutes, the living cell count of the respective cultures was determined. To this end, samples corresponding to an OD₅₈₀ of 0.1 were removed from the cultures. Next, several dilutions (1:5000, 1:50000; 1:75000 and 1:100000) were plated out onto LB solid medium and incubated overnight under selection pressure at 37° C. The count of the colonies grown on these plates was then determined and the ratio of living cells after 90 minutes irradiation was calculated therefrom. The values are given in Table 1.

TABLE 1
Ratio of living cell count of E. coli DH5α cultures after blue light
irradiation (λ = 460 nm) for 90 minutes to the living cell count
immediately before blue light irradiation

| pRhokHi-2 empty vector | pRhokHi-2_SB2 | pRhokHi-2_PpFbFP |
|---|---|---|
| 77% | 92% | 0% |

The living cell count determination clearly shows that irradiation with blue light, which is specifically absorbed by the chromophore of the LOV protein, has a phototoxic effect on the E. coli cells which is brought about by the FbFP with SEQ ID No. 6. However, the phototoxic effect was not observed for either the SB2-expressing E. coli cultures, nor for the empty vector control.

In addition, it was clear that illumination of the E. coli cultures which carry only the empty vector instead of the FbFP with SEQ ID No. 6 expression vector, does not experience any growth inhibition with blue or red light. This is also the case for SB2-expressing strains; their growth curves, which are also independent of the light exposure, are almost identical.

4. Dependency of Toxic Effect of FbFP with SEQ ID No. 6 with Time.

In order to better resolve the blue light-dependent toxic effect of FbFP with SEQ ID No. 6 with time, the living cell count was determined as a function of the illumination period. To this end, 5 mL of a LB pre-culture was inoculated with an appropriate colony from a LB solid medium plate and incubated overnight on a rotary shaker under selection pressure at 37° C. From this pre-culture, a 50 mL LB test culture (50 μg/mL kanamycin) was inoculated with an OD₅₈₀ of 0.05 and cultured with agitation in the dark at 37° C. Again, a test culture of E. coli cells which had been transformed with the pRhokHi-2 empty vector acted as the negative control. In analogy with the preceding experiments, the test cultures were subjected to direct blue light irradiation (λ=460 nm) starting 2 hours after inoculation; cell samples (OD₅₈₀=0.1) were taken at regular intervals. Dilutions of the cell samples were made at factors 1:5000, 1:50000, 1:75000 and 1:100000 and spread onto respective LB solid medium plates and then grown overnight at 37° C. under selection pressure. Next, the colony forming units (CFU) were determined by counting the plates and referring it to the respective CFU count prior to irradiation.

These curves show that the living cell count of the FbFP with SEQ ID No. 6-expressing E. coli culture left in darkness and the two empty vector controls remained almost constant, independently of the illumination conditions. In contrast, a significant reduction in the living cell count of the culture which expresses FbFP with SEQ ID No. 6 was observed for long-duration blue light irradiation. After just 10 minutes irradiation, the living cell count had reduced by about half; after 30 minutes irradiation, almost all of the cells of the culture had been killed off.

5. New Demonstration of Phototoxic Effect of FbFP with SEQ ID No. 6.

In order to elucidate again the demonstrated phototoxic effect of blue light on E. coli cells which express FbFP with SEQ ID No. 6 and the possible use of FbFP with SEQ ID No. 6 as a photosensitizer associated therewith, a sample of the pre-cultures of E. coli (pRhokHi-2_PpFbFP) strain grown overnight, as well as the empty vector control were spread onto LB solid medium (50 μg/mL kanamycin) and incubated overnight under blue light irradiation or in the dark at 37° C. The results once again impressively demonstrated the phototoxic effect of FbFP with SEQ ID No. 6. Thus, the growth of the empty vector control was not affected by irradiation with blue light. On the other hand, the FbFP with SEQ ID No. 6-expressing strain did not grow under blue light, whereas the growth in darkness was unaffected. In order to check once again whether the toxic effect is bactericidal or bacteriostatic in nature, one of the plates used was then incubated in the dark for a further night at 37° C. Since the number and distribution of the colonies did not change, then the conclusion can be drawn that it is a bactericidal effect.

The results presented here clearly show that FbFP with SEQ ID No. 6 exerts a phototoxic effect on E. coli cells which is specifically induced by blue light.

6. Use of FbFP with SEQ ID No. 6 for Chromophore-Assisted Light Inactivation (CALI) of Target Proteins

In order to check whether the blue light photosensitizer FbFP with SEQ ID No. 6 could also be used for CALI-mediated inactivation of fusion proteins, FbFP with SEQ ID No. 6 was fused to the yellow fluorescent protein YFP (SEQ ID No. 9). This was carried out in order to check whether it was possible to inactivate the activity of the YFPs by means of the FbFP with SEQ ID No. 6-mediated CALI reaction.

To this end, translation fusions were generated in which the photosensitizer FbFP with SEQ ID No. 6 was fused to the C-terminal end of the target protein YFP. The proteins were bonded by means of a linker (SEQ ID No. 10 and 11), which contains a “multiple cloning site” (MCS) with the cleavage sites for the restriction endonucleases KpnI, NdeI, BamHI, SacI, SalI, HindIII, XhoI and Cfr42I. To express the fusion protein, the expression vector pRhotHi-2 was again selected. The fusion protein was also provided with a His₆ tag, which meant that the recombinant protein could be readily purified by affinity chromatographic techniques.

All of the cloning steps were carried out in the cloning vector pBlueScript KSII(−). The cloning strategy is diagrammatically illustrated in FIG. 9, FIG. 10 and FIG. 11. The respective complete recombinant genes were cut out of the vector following successful sequencing by restriction digestion and then cloned into the expression vector pRhotHi-2.

Next, the fusion protein was over-expressed in E. coli using the His₆-tag, purified by means of Ni-NTA-affinity chromatography and taken up in a protein buffer (10 mM NaH₂PO₄, 10 Mm NaCl, pH 8). In order to check whether the YFP reporter protein can be specifically inactivated by FbFP with SEQ ID No. 6 by means of CALI, both the fusion protein and the non-fused YFP protein were irradiated for a period of 60 min in blue light (λ=488 nm) and then the YFP-specific fluorescence was determined as a measure of the target protein activity.

As can be seen in FIG. 11, irradiation with blue light of the YFP-FbFP with SEQ ID No. 6 fusion resulted in a rapid reduction in the YFP fluorescence. The YFP protein is thus FbFP with SEQ ID No. 6-dependently inactivated by the CALI method. In contrast to this, the blue light irradiation had no effect on the activity of the non-fused YFP protein. Thus, it can clearly be seen that the reduction of YFP fluorescence in the case of the fusion protein is exclusively due to the light-inactivating effect of FbFP with SEQ ID No. 6. A fresh measurement of the YFP fluorescence after 24 h of incubation of the samples which had been irradiated with blue light also showed that the observed FbFP with SEQ ID No. 6-mediated inactivation of YFP is irreversible, since no regeneration of the fluorescence could be detected. Thus, the novel photosensitizer FbFP with SEQ ID No. 6 can be used not only to kill off individual cells, but also for specific inactivation of any target proteins inside and outside cells.

TABLE 2

| SEQ | | |
|---|---|---|
| ID No | Name | Sequence |
| 1 | LOV domain | MASFQSFGIP GQLEVIKKAL DHVRVGVVIT |
| | from Bacillus | DPALEDNPIV YVNQGFVQMT GYETEEILGK |
| | subtilis | NARFLQGKHT DPAEVDNIRT ALQNKEPVTV |
| | | QIQNYKKDGT MFWNELNIDP MEIEDKTYFV |
| | | GIQNDITKQK EYEKLLEDSL TEITALSTPI |
| | | VPIRNGISAL PLVGNLTEER FNSIVCTLTN |
| | | ILSTSKDDYL IIDLSGLAQV NEQTADQIFK |
| | | LSHLLKLTGT ELIITGIKPE LAMKMNKLDA |
| | | NFSSLKTYSN VKDAVKVLPI M |
| 2 | LOV domain | MASFQSFGIP GQLEVIKKAL DHVRVGVVIT |
| | from Bacillus | DPALEDNPIV YVNQGFVQMT GYETEEILGK |
| | subtilis | NARFLQGKHT DPAEVDNIRT ALQNKEPVTV |
| | | QIQNYKKDGT MFWNELNIDP MEIEDKTYFV |
| | | GIQNDITKQK EYEKLLE |
| 3 | LOV domain | MINAQLLQSM VDASNDGIVV AEKEGDDTIL |
| | from | IYVNAAFEYL TGYSRDEILY QDARFLQGDD |
| | Pseudomonas | RDQLGRARIR KAMAEGRPCR EVLRNYRKDG |
| | putida | SAFWNELSIT PVKSDFDQRT YFIGIQKDVS |
| | | RQVELERELA ELRARPKPDE RA |
| 4 | LOV domain | MINAKLLQLM VEHSNDGIVV AEQEGNESIL |
| | from | IYVNPAFERL TGYCADDILY QDARFLQGED |
| | Pseudomonas | HDQPGIAIIR EAIREGRPCC QVLRNYRKDG |
| | putida | SLFWNELSIT PVHNEADQLT YYIGIQRDVT |
| | | AQVFAEERVR ELEAEVAELR RQQGQAKH |
| 5 | FbFP nucleotide | atgatcaacg caaaactcct gcaactgatg gtcgaacatt ccaacgatgg |
| | sequence | catcgttgtc gccgagcagg aaggcaatga gagcatcctt atctacgtca |
| | | acccggcctt cgagcgcctg accggctact gcgccgacga tattctctat |
| | | caggacgccc gttttcttca gggcgaggat cacgaccagc |
| | | cgggcatcgc aattatccgc gaggcgatcc gcgaaggccg |
| | | cccctgctgc caggtgctgc gcaactaccg caaagacggc agcctgttct |
| | | ggaacgagtt gtccatcaca ccggtgcaca acgaggcgga |
| | | ccagctgacc tactacatcg gcatccagcg cgatgtcaca gcgcaagtat |
| | | tcgccgagga aagggttcgc gagctggagg ctgaagtggc |
| | | ggaactgcgc cggcagcagg gccaggccaa gcactga |
| 6 | FbFP AA | MINAKLLQLM VEHSNDGIVV AEQEGNESIL |
| | sequence | IYVNPAFERL TGYCADDILY QDARFLQGED |
| | | HDQPGIAIIR EAIREGRPCC QVLRNYRKDG |
| | | SLFWNELSIT PVHNEADQLT YYIGIQRDVT |
| | | AQVFAEERVR ELEAEVAELR RQQGQAKH |
| 7 | LOV-consensus | NCRFLQ |
| | sequence | |
| 8 | YFP nucleotide | ATGGTGAGCAAGGGCGAGGAGCTGTTCACCGG |
| | sequence | GGTGGTGCCCATCCTGGTCGAGCTGGACGGCG |
| | | ACGTAAACGGCCACAAGTTCAGCGTGTCCGGC |
| | | GAGGGCGAGGGCGATGCCACCTACGGCAAGCT |
| | | GACCCTGAAGTTCATCTGCACCACCGGCAAGCT |
| | | GCCCGTGCCCTGGCCCACC |
| | | CTCGTGACCACCTTCGGCTACGGCCTGCAGTGC |
| | | TTCGCCCGCTACCCCGACCACATGAAGCAGCAC |
| | | GACTTCTTCAAGTCCGCCATGCCCGAAGGCTAC |
| | | GTCCAGGAGCGCACCATCTTCTTCAAGGACGAC |
| | | GGCAACTACAAGACCCGCGCCGAGGTGAAGTT |
| | | CGAGGGCGACACCCTG |
| | | GTGAACCGCATCGAGCTGAAGGGCATCGACTT |
| | | CAAGGAGGACGGCAACATCCTGGGGCACAAGC |
| | | TGGAGTACAACTACAACAGCCACAACGTCTAT |
| | | ATCATGGCCGACAAGCAGAAGAACGGCATCAA |
| | | GGTGAACTTCAAGATCCGCCACAACATCGAGG |
| | | ACGGCAGCGTGCAGCTCGCC |
| | | GACCACTACCAGCAGAACACCCCCATCGGCGA |
| | | CGGCCCCGTGCTGCTGCCCGACAACCACTACCT |
| | | GAGCTACCAGTCCGCCCTGAGCAAAGACCCCA |
| | | ACGAGAAGCGCGATCACATGGTCCTGCTGGAG |
| | | TTCGTGACCGCCGCCGGGATCACTCTCGGCATG |
| | | GACGAGCTGTACAAGTAA |
| 9 | YFP peptide | MVSKGEELFTGVVPILVELDGDVNGHKFSVSGEG |
| | sequence | EGDATYGKLTLKFICTTGKLPVPWPTLVTTFGYG |
| | | LQCFARYPDHMKQHDFFKSAMPEGYVQERTIFFK |
| | | DDGNYKTRAEVKFEGDTLVNRIELKGIDFKEDGN |
| | | ILGHKLEYNYNSHNVYIMADKQKNGIKVNFKIRH |
| | | NIEDGSVQLA |
| | | DHYQQNTPIGDGPVLLPDNHYLSYQSALSKDPNE |
| | | KRDHMVLLEFVTAAGITLGMDELYK |
| 10 | Upper Primer | ggtacccata tgggatccga gctcgcgggc ctggtgccgc |
| | Linker | gcggcagcgg |
| 11 | Down Primer | ccgcggctcg agaagcttgt cgacggcgcc gctgccgcgc |
| | Linker | ggcaccaggc |
| 12 | SB2 + NdeI-up | atcgcgcata tgatcaacgc aaaactc |
| 13 | SB2 + XhoI-down | cgtctcgagt cagtgcttgg cctggc |

### LITERATURE

Jacobson, K., Rajfur, Z., Vitriol, E. & Hahn, K. (2008). Chromophore-assisted laser inactivation in cell biology. Trends Cell Biol 18: 443-450.

1. Use of a fluorescence protein comprising a LOV domain for the photosensitive defunctionalization of a molecule, wherein at least one cysteine in the LOV domain is replaced by another amino acid which does not covalently bind any FMN.

2. Use of a protein as claimed in claim 1, in a solution or a cell.

3. Use of a protein as claimed in claim 1, wherein the photosensitive defunctionalization is chromophore assisted light inactivation (CALI) and/or a phototoxic reaction.

4. Use of a protein as claimed in wherein said LOV domain
a) is encoded by the nucleic acids of SEQ ID No. 5 or a fragment, a variant, a homologue or a derivative of this sequence,
b) is encoded by a nucleic acid which can hybridize with the nucleic acids from a) under stringent conditions,
c) is encoded by a nucleic acid which has at least 70%, preferably 95% identity with one of the nucleic acids from a) or b),
d) is encoded by a nucleic acid which can hybridize under stringent conditions with the complementary nucleic acid of one of the nucleic acids from a)-c),
e) is encoded by a nucleic acid which, compared with the nucleic acids from a)-d), has at least one silent mutation of a single nucleotide (as allowed by the degeneration of the genetic code),
f) is encoded by a nucleic acid the code for which has been optimized for a specific expression system compared with the nucleic acids from a)-e),
g) comprises an amino acid sequence in accordance with SEQ ID No. 6 or a fragment, a variant, a homologue or a derivative of this sequence,
h) comprises an amino acid sequence which has a sequence identity of at least 70%, preferably 95% with the amino acid sequences from g).

5. A method for the photosensitive defunctionalization of a target molecule, comprising at least the following steps:
introducing a vector which encodes a protein which is photoactive at wavelengths of 380-490 nm into a cell which contains the target molecule, and expressing the protein in this cell, or coupling a protein which is photoactive at wavelengths of 380-490 nm to a target molecule irradiating the cell or the protein-target molecule complex with light with wavelengths of 380-490 nm.

6. The method as claimed in claim 5, additionally comprising the following steps before coupling the protein to the target molecule:
introducing a vector into a cell, which vector encodes the protein which is photoactive at wavelengths of 380-490 nm,
expressing the protein in the cell
extracting the protein.

7. The method as claimed in claim 5, additionally comprising the step in which the protein-target molecule complex is introduced into a cell before irradiation.

8. The method as claimed in claim 5, wherein the protein and the target molecule can be expressed together as a transcription unit.

9. The method as claimed in claim 5, wherein the cell which expresses the protein is preferably a bacterium selected from the group consisting of Escherichia coli, Rhodobacter capsulatus, Pseudomonas putida and/or Bacillus subtilis.

10. The method as claimed in claim 6, additionally comprising the step in which the protein-target molecule complex is introduced into a cell before irradiation.

11. The method as claimed in claim 6, wherein the protein and the target molecule can be expressed together as a transcription unit.

12. The method as claimed in claim 6, wherein the cell which expresses the protein is preferably a bacterium selected from the group consisting of Escherichia coli, Rhodobacter capsulatus, Pseudomonas putida and/or Bacillus subtilis.

13. The method as claimed in claim 7, wherein the protein and the target molecule can be expressed together as a transcription unit.

14. The method as claimed in claim 7, wherein the cell which expresses the protein is preferably a bacterium selected from the group consisting of Escherichia coli, Rhodobacter capsulatus, Pseudomonas putida and/or Bacillus subtilis.

15. The method as claimed in claim 6, additionally comprising the step in which the protein-target molecule complex is introduced into a cell before irradiation, wherein the protein and the target molecule can be expressed together as a transcription unit, and wherein the cell which expresses the protein is preferably a bacterium selected from the group consisting of Escherichia coli, Rhodobacter capsulatus, Pseudomonas putida and/or Bacillus subtilis.


SEQUENCE LISTING (13 sequences)

SEQ ID NO: 1 - protein, 261 aa, Bacillus subtilis
MASFQSFGIPGQLEVIKKALDHVRVGVVITDPALEDNPIVYVNQGFVQMTGYETEEILGKNARFLQGKHTDPAEVDNIRTALQNKEPVTVQIQNYKKDGTMFWNELNIDPMEIEDKTYFVGIQNDITKQKEYEKLLEDSLTEITALSTPIVPIRNGISALPLVGNLTEERFNSIVCTLTNILSTSKDDYLIIDLSGLAQVNEQTADQIFKLSHLLKLTGTELIITGIKPELAMKMNKLDANFSSLKTYSNVKDAVKVLPIM

SEQ ID NO: 2 - protein, 137 aa, Bacillus subtilis
MASFQSFGIPGQLEVIKKALDHVRVGVVITDPALEDNPIVYVNQGFVQMTGYETEEILGKNARFLQGKHTDPAEVDNIRTALQNKEPVTVQIQNYKKDGTMFWNELNIDPMEIEDKTYFVGIQNDITKQKEYEKLLE

SEQ ID NO: 3 - protein, 142 aa, Pseudomonas putida
MINAQLLQSMVDASNDGIVVAEKEGDDTILIYVNAAFEYLTGYSRDEILYQDARFLQGDDRDQLGRARIRKAMAEGRPCREVLRNYRKDGSAFWNELSITPVKSDFDQRTYFIGIQKDVSRQVELERELAELRARPKPDERA

SEQ ID NO: 4 - protein, 148 aa, Pseudomonas putida
MINAKLLQLMVEHSNDGIVVAEQEGNESILIYVNPAFERLTGYCADDILYQDARFLQGEDHDQPGIAIIREAIREGRPCCQVLRNYRKDGSLFWNELSITPVHNEADQLTYYIGIQRDVTAQVFAEERVRELEAEVAELRRQQGQAKH

SEQ ID NO: 5 - DNA, 447 bp, Pseudomonas putida
atgatcaacgcaaaactcctgcaactgatggtcgaacattccaacgatggcatcgttgtcgccgagcaggaaggcaatgagagcatccttatctacgtcaacccggccttcgagcgcctgaccggctactgcgccgacgatattctctatcaggacgcccgttttcttcagggcgaggatcacgaccagccgggcatcgcaattatccgcgaggcgatccgcgaaggccgcccctgctgccaggtgctgcgcaactaccgcaaagacggcagcctgttctggaacgagttgtccatcacaccggtgcacaacgaggcggaccagctgacctactacatcggcatccagcgcgatgtcacagcgcaagtattcgccgaggaaagggttcgcgagctggaggctgaagtggcggaactgcgccggcagcagggccaggccaagcactga

SEQ ID NO: 6 - protein, 148 aa, Pseudomonas putida; identical to SEQ ID NO: 4

SEQ ID NO: 7 - protein, 6 aa, Artificial
  Consensus sequence
NCRFLQ

SEQ ID NO: 8 - DNA, 720 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 9 - protein, 239 aa, Aequorea victoria
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 10 - DNA, 50 bp, Artificial
  Upper Primer Linker
ggtacccatatgggatccgagctcgcgggcctggtgccgcgcggcagcgg

SEQ ID NO: 11 - DNA, 50 bp, Artificial
  Down Primer Linker
ccgcggctcgagaagcttgtcgacggcgccgctgccgcgcggcaccaggc

SEQ ID NO: 12 - DNA, 27 bp, Artificial
  SB2+NdeI-up
atcgcgcatatgatcaacgcaaaactc

SEQ ID NO: 13 - DNA, 26 bp, Artificial
  SB2+XhoI-down
cgtctcgagtcagtgcttggcctggc